(19) United States
(12) Patent Application Publication
(10) Pub. No.: US 2010/0249501 A1
(43) Pub. Date: Sep. 30, 2010

(54) ENDOSCOPE APPARATUS

(76) Inventors: Kiyotaka Matsuno, Tokyo (JP); Takumi Dejima, Tokyo (JP); Manabu Miyamoto, Tokyo (JP); Ryo Minosawa, Tokyo (JP); David Edward Barlow, Coopersburg, PA (US)

Correspondence Address: OSTROLENK FABER GERB & SOFFEN, 1180 AVENUE OF THE AMERICAS, NEW YORK, NY, 100368403, US

(21) Appl. No.: 12/593,064

(22) PCT Filed: Mar. 27, 2007
(86) PCT No.: PCT/JP2007/057345

§ 371 Date: May 20, 2010

(51) Int. Cl. A61B 1/04

(52) U.S. Cl. 600/109

(57) ABSTRACT

The endoscope apparatus comprises an endoscope which has an imaging element and a monitor which displays an image imaged by the imaging element. The endoscope is provided with an internal insertion portion which is movably arrangeable inside a body cavity, and an operation unit for changing the position of the internal insertion portion, which is arrangeable outside the body. The internal insertion portion and the operation unit are arranged on a common axis so as to interpose a body wall therebetween.

### TECHNICAL FIELD

The present invention relates to an endoscope apparatus, in particular, an endoscope apparatus which moves a treating unit or an observation unit which has an imaging element and which is movably arrangeable inside of a body cavity, using an operation unit which is arranged outside of the body so as to interpose a body wall with the treating unit or the observation unit.

### BACKGROUND ART

In conventional endoscopic surgical operation, various operations are commonly performed using a plurality of forceps and a rigid endoscope inserted into the body cavity through a plurality of openings having 3 to 10 mm diameter made in a body wall.

Japanese Unexamined Patent Application, First Publication No. H11-326783 discloses a rigid endoscope used in such surgery.

The rigid endoscope comprises a rigid insertion tube and a monitor connected to a base end of the rigid insertion tube. An object lens system, an imaging element, and a signal cable extending from the imaging element are built in the rigid insertion tube in this order from a tip of the rigid insertion tube. The monitor is electrically connected to the imaging element through the signal cable. The tip of the rigid insertion tube of the rigid endoscope is inserted through one of the openings made in the body wall, and the image of the inside of the body cavity picked up by the imaging element is displayed on the monitor.

In addition, Japanese Unexamined Patent Application, First Publication No. 2003-1117220 discloses an endoscope used in surgery provided with an internal robot and a 3-D gradient magnetic field generator. The internal robot of this apparatus is inserted into the body cavity and operated by using remote control method by means of the magnetism produced by the 3-D gradient magnetism generator. Then, the images of the inside of the body cavity are picked up by the imaging element attached in the internal robot, and the images are transmitted to outside of the body using a transmitting device built in the internal robot.

Actually, the inserted rigid endoscope is susceptible to limitation in an endoscopic surgical operation using both the rigid endoscope and forceps because the body cavity formed in the body wall limits the movable region of the rigid endoscope. Therefore, a diseased part can be observed from the limited directions. Also, an image obtained by a rigid endoscope and displayed on the monitor can hardly provide instantaneous recognition as to which direction the monitored image is picked up from.

Significance of the 3-D gradient magnetic field generator in size used together with the internal robot requires a large space in surgery. The 3-D gradient magnetic field generator further adds to facility cost and operational cost.

The present invention was conceived in consideration of the aforementioned circumstances, and an object thereof is to provide an endoscope apparatus operable easily and capable of observing a diseased part in an arbitrary direction in well-visualized manner without extensive facility.

### DISCLOSURE OF INVENTION

An endoscope apparatus according to the present invention includes: an endoscope which has an imaging element; and a monitor which displays an image imaged by the imaging element; wherein the endoscope is provided with an internal insertion portion which is movably arrangeable inside a body cavity, and an operation unit for changing the position of the internal insertion portion, which is arrangeable outside the body, and wherein the internal insertion portion and the operation unit are arranged on a common axis so as to interpose a body wall therebetween.

The endoscope apparatus provides recognizable vision of a diseased part in an arbitrary direction in an endoscopic surgical operation. Also, the present invention provides easy operability while suppressing facility cost and operational cost because extensive facility is not necessary.

### BRIEF DESCRIPTION OF DRAWINGS

FIG. 1 is a schematic view of an endoscope apparatus according to the first embodiment of the present invention.

FIG. 2 is an enlarged sectional view of a part of the endoscope apparatus in use according to the first embodiment of the present invention.

FIG. 3 is a schematic plan view showing the endoscope apparatus according to the second embodiment of the present invention.

FIG. 4 is a sectional view taken along a line A-A of FIG. 3, showing the endoscope apparatus according to the second embodiment of the present invention.

FIG. 5 is an enlarged sectional view of a part of FIG. 4, showing the endoscope apparatus according to the second embodiment of the present invention.

FIG. 6 is a sectional view taken along a line B-B of FIG. 5 showing the endoscope apparatus according to the second embodiment of the present application.

FIG. 7 is a view taken along an arrow C of FIG. 3 showing the endoscope apparatus according to the second embodiment of the present invention.

FIG. 8 is a sectional view taken along a line D-D of FIG. 7 showing the endoscope apparatus according to the second embodiment of the present invention.

FIG. 9 is an enlarged view of a part E indicated in FIG. 3, showing the endoscope apparatus according to the second embodiment of the present invention.

FIG. 10 is a schematic view of an endoscope apparatus according to the first embodiment of the present invention.

FIG. 11 is a sectional view, showing the state of use of the endoscope apparatus according to the third embodiment of the present invention.

FIG. 12 is a schematic view of an endoscope apparatus according to the first embodiment of the present invention.

FIG. 13 is a schematic view of an endoscope apparatus according to the first embodiment of the present invention.

FIG. 14 illustrates the endoscope apparatus according to a fifth embodiment of the present invention viewed along a line F-F of FIG. 13.

FIG. 15 illustrates the endoscope apparatus according to the fifth embodiment of the present invention viewed along a line G-g of FIG. 13.

### BEST MODE FOR CARRYING OUT THE INVENTION

Preferred embodiments of the present invention will be explained hereafter, with reference to the drawings. Same reference numerals are added to same components in the drawings regardless of the embodiments, and duplicate explanations are omitted.

### First Embodiment

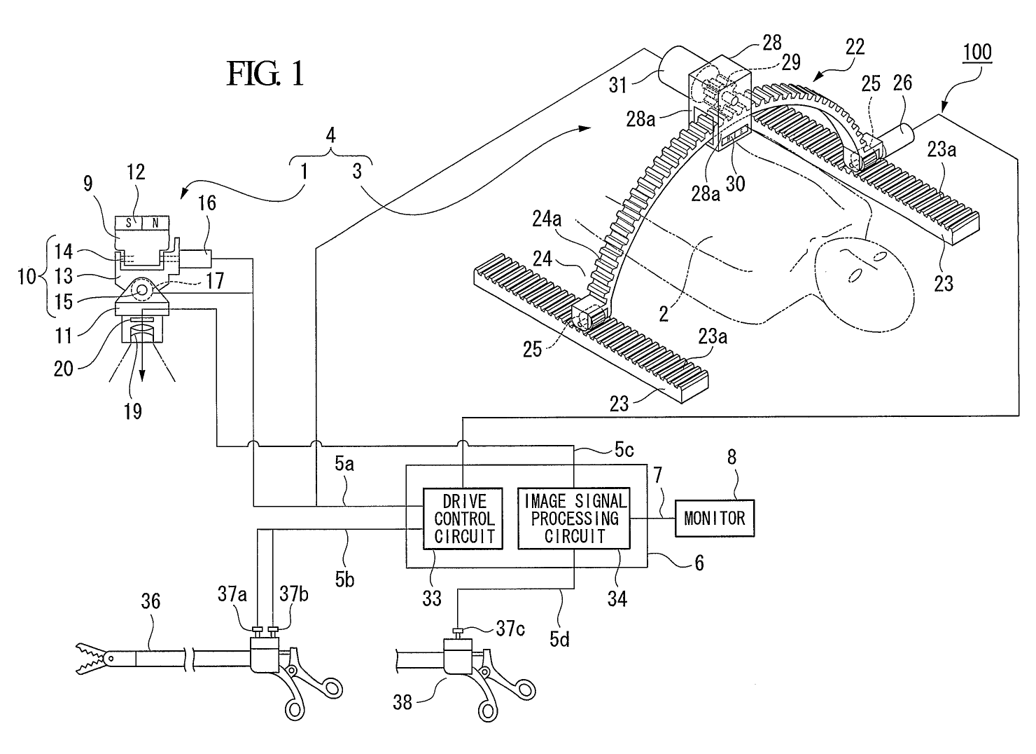

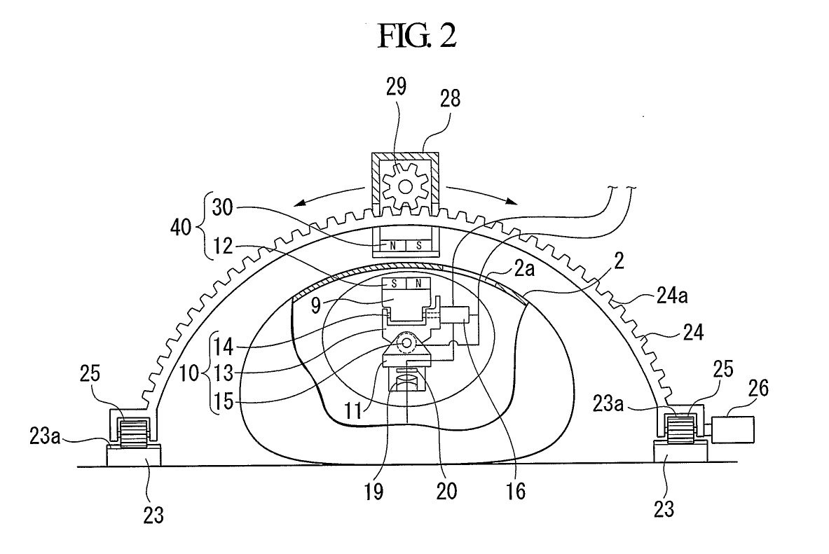

An endoscope apparatus according to the first embodiment of the present invention will be hereafter explained with reference to FIGS. 1 and 2.

FIG. 1 is a schematic view of the endoscope apparatus according to the first embodiment of the present invention. An endoscope apparatus 100 includes an endoscope 4; a processor 6 which is electrically connected to the endoscope 4 through transmission wires 5a and 5c, and a monitor 8 which is electrically connected to a processor 6 through a transmission wire 7 and which displays an image picked up by the endoscope 4. The endoscope 4 includes an internal insertion portion 1 which is movably arranged inside of the body cavity, and an operation unit 3 which is movably arranged outside of the body so as to interpose a body wall 2 with the internal insertion portion 1, and which changes the position of the internal insertion portion 1.

The internal insertion portion 1 includes a base portion 9, and an observation unit main body 11 which is attached to the base portion 9 through a joint portion 10 so that the angle of the observation unit main body 11 relative to the base portion 9 is variable. A magnet 12 is attached to a body wall side end portion of the internal insertion portion 1 arranged inside of the body cavity. For example, the magnet 12 may be a permanent magnet having a rectangular shape.

The joint portion 10 includes: an intermediate body 13 which is disposed between the base portion 9 and the observation unit main body 11; a rotation shaft 14 which is attached to the base portion 9 and the intermediate body 13; and a rotation shaft 15 which is attached to the intermediate body 13 and the observation unit main body 11. The rotation shaft 14 and the rotation shaft 15 are arranged so as to mutually cross. The rotation shaft 14 is connected to a motor 16, and the rotation shaft 15 is connected to a motor 17. The rotation shaft 14 is rotated in both forward and reverse directions by the motor 16, and the rotation shaft 15 is rotated in both forward and reverse directions by the motor 17.

The observation unit main body 11 includes an object optical system 19 and an imaging element 20.

The operation unit 3 guided by a guide portion 22 is moved along the body wall 2, and is fixed on a predetermined position. The guide portion 22 includes two rectilinear rails 23 which are disposed parallel to each other, and an arched rail 24 which is disposed between the two rectilinear rails 23 and which is movable along the two rectilinear rails 23. The arched rail 24 movably guides the operation unit 3 along an arched path thereof. Pinions 25 are attached to both ends of the arched rail 24, and engage racks 23a are formed on upper surfaces of the two rectilinear rails 23. The arched rail 24 moves along the rectilinear rails 23 while of crossing the rectilinear rails 23 by synchronously rotating both the pinions 25 by a motor 26.

Note that, FIG. 1 shows only the connection of the motor 26 to one of the pinions 25; a different motor is connected to the other pinion 25.

The operation unit 3 includes: a housing 28; a pinion 29 which is rotatably supported by the housing 28, and which engages with a teeth portion 24a formed on an upper surface of the arched rail 24; and a magnet 30 which is attached to a lower part of the housing 28. The housing 28 is provided with side portions 28a, 28a which interpose both sides of the arched rail 24. The pinion 29 rotated by the motor 31 guides the operation unit 3 along the arched rail 24.

A coupler 40 follows the operation unit 3 moved by the guide portion 22 along the body wall 2 and moves the internal insertion portion 1 inside the body cavity so that the internal insertion portion 1 and the operation unit 3 are directed to each other on both sides of the body wall 2. The coupler 40 includes the magnet 12 which is disposed on the operation unit 3, and the magnet 30 which is disposed on the internal insertion portion 1 (Refer to FIG. 2).

In addition, the magnet 30 of the operation portion 3 has a magnetic force for lifting up the internal insertion portion 1 toward the body wall 2 through the magnet 12 which is directed toward the magnet 30 across the body wall 2. The magnet 30 may be an electromagnet or a superconductive magnet having greater magnetic force. The same applies to the magnet 12 disposed on the base 9.

The magnet 30 of the operation unit 3 and the magnet 12 of the internal insertion portion 1 are positioned so that the rotation shaft 14 is parallel to a surface of the arched rail 24, and the rotation shaft 15 crosses the surface of the arched rail 24 when the magnet 30 of the operation unit 3 absorbs the magnet 12 of the internal insertion portion 1 in a regular state interposing the body wall 2.

Note that, the configuration is not limited to examples explained with reference to the drawings in which the arched rail 24 is moved relative to the rectilinear rails 23 by assembling the pinions 25 to the engage rack 23a; and the arched rail 24 may also be moved relative to the rectilinear rails 23 by means of a ball screw mechanism. In addition, the ball screw mechanism may also be used for moving the operating unit 3 along the arched rail 24.

The processor 6 includes a drive control circuit 33 and an image signal processing circuit 34.

The drive control circuit 33 is electrically connected to the motors 16, 17, 26 through the transmission wire 5a, and a motor 31 is electrically connected to switches (inputting unit) 37a, 37b of rigid forceps 36 through a transmission wire 5b. The image signal processing circuit 34 is electrically connected to the imaging element 20 through the transmission wire 5c, and electrically connected to a zoom switch (inputting unit) 37c of rigid forceps 38 through a transmission wire 5d. In addition, the image signal processing circuit 34 is electrically connected to the monitor 8 through the transmission wire 7.

The switch 37a used here is a four-directional inputting switch such as a joystick or the like. For example, actuating the switch 37a in a lateral direction drives the motor 31, thereby moving operation unit 3 along the arched rail 24. Actuating in a back and forth direction drives the motor 26, thereby moves the arched rail 24 relative to the rectilinear rail 23 by means of drive control circuit 33. The switch 37b is also a four-directional inputting switch such as a joystick or the like. For example, actuating the switch 37 in a lateral direction drives the motor 16, thereby rotating intermediate body 13 relative to the base portion 9 by driving the motor 17. Actuating in a back and forth direction drives the motor 17, thereby rotating the observation unit main body 11 relative to the intermediate body 13 by means of drive control circuit 33.

In addition, the zoom switch 37c electrically controls image zooming. Actuating the switch 37c drives the image signal processing circuit 34, thereby enlarging and reducing the image in size.

The operation of the endoscope apparatus 100 having the aforementioned configuration will be explained next.

As shown in FIG. 1, the arched rail 24 and the rectilinear rails 23 are disposed along the body wall 2 of a patient.

Next, the internal insertion portion 1 is inserted into the body cavity through the opening 2a formed in the body wall 2, and is brought close to the operating unit 3 previously fixed to the arched rail 24. The absorbing force between the magnet 30 of the operation unit 3 and the magnet 12 of the internal insertion portion 1 presses and fixes the internal insertion portion 1 to innermore of the body wall 2.

The information obtained by the observation unit main body 11 of the internal insertion portion 1 is projected on the monitor 8 through the image signal processing circuit 34. An operator viewing the monitor 8 treats the patient by operating the rigid forceps 36, 38, which have been inserted through a different opening 2a in the body wall 2.

For example, actuating the joystick switch 37a attached to the rigid endoscope 36 controls the movement of the operation unit 3 along the arched rail 24, and the movement of the arched rail 24 along the rectilinear rails 23, thereby providing the side view of the diseased part. At this time, the internal insertion portion 1 linked with the operation unit 3 by magnetic force then moves inside of the operation unit 3. Actuating further the switch 37b drives the motors 16 and 17 respectively, thereby adjusting the inclination angle of the observation unit main body 11 relative to the base 9.

This allows the operator to treat a diseased part while observing the diseased part from a desirable angle.

Similarly, actuating the zoom switch 37c attached to the rigid forceps 38 enables the treatment of diseased part together with the visual observation of desired image in size on the monitor 8 by means of the image signal processing circuit 34.

Since the internal insertion portion 1 is movable in the endoscope apparatus having the above configuration in which the internal insertion portion 1 and the operation unit 3 placing the body wall 2 therebetween are fixed by magnetic force, and the movement of the operation unit 3 outside the body allows the movement of the internal insertion portion 1. Therefore, it is possible to move the internal insertion portion 1 relative to the body wall 2 without restriction of the position of the opening 2a made in the body wall 2, and it is possible to observe the diseased part from any direction.

In addition, since the internal insertion portion 1 is attached to the operation unit 3 so that the body wall 2 is interposed between the internal insertion portion 1 and the operation unit 3, the position of the operation unit 3 is roughly equal with the position of the internal insertion portion 1, that is the position of the observation unit main body 11. Therefore, it is easy to intuitively understand the position from which the image displayed on the monitor 8 is captured, and it is easy to perform orientation. As a result, it is possible to treat the diseased part appropriately.

Further, according to the endoscope apparatus as described above, since it is not necessary to use a large-size magnetic field generator, it is possible to reduce the space and number of components required, thereby achieving low-cost.

In conventional surgery using an endoscope apparatus, an operator who operates a rigid endoscope known as a “scopist”, and another operator who operates two rigid forceps are required. However, according to the endoscope apparatus as described above, an operator who operates the two rigid forceps 36, 38 is able to move the internal insertion portion 1 and to operate zooming. Therefore, only one operator is required to operate the forceps 36, 38 and the endoscope, and the above mentioned “scopist” is not required.

### Second Embodiment

An explanation of the endoscope apparatus relating to the second embodiment of the present invention is described hereafter.

The second embodiment differs from the above-mentioned first embodiment of the present invention as follows. That is, while the operation unit 3 is guided along the body wall 2 by the guide portion 22, a coupler 40 moves the internal insertion portion 1 following the movement of the operation unit 3 so that the internal insertion portion 1 and the operation unit 3 are arranged on a common axis so as to interpose the body wall 2 therebetween.

In the first embodiment, the operation unit 3 is moved by the motors 26, 31. However, in the second embodiment, the operation unit 3 is moved manually without the driving force of the motor or the like. Therefore, the endoscope apparatus of the second embodiment does not include the motors 26, 31. In this point as well, the second embodiment differs from the first embodiment.

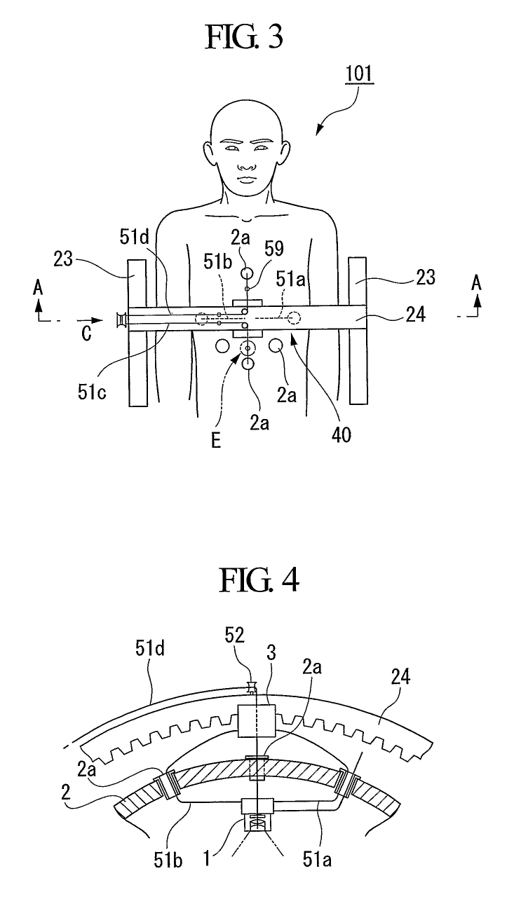

An explanation of the endoscope apparatus relating to the second embodiment is described hereafter, with reference to FIGS. 3 to 9.

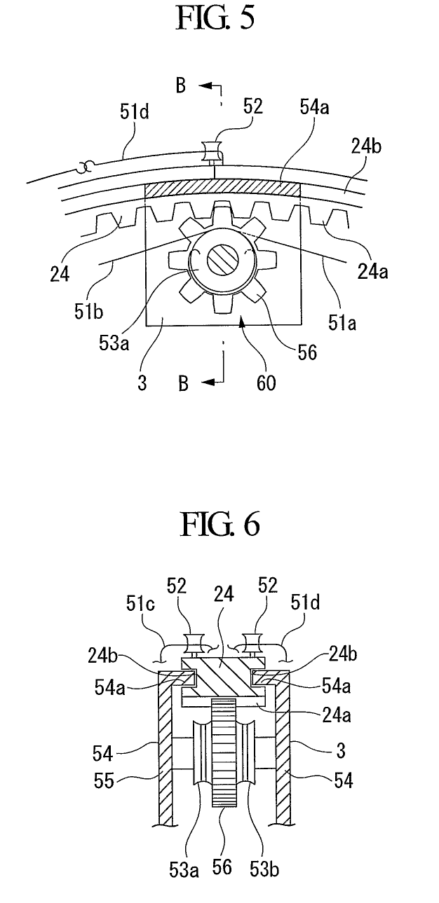

FIG. 3 is a schematic plan view showing an endoscope apparatus 101. FIG. 4 is a sectional view taken along a line A-A of FIG. 3, FIG. 5 to FIG. 9 are enlarged sectional views of an essential part of the endoscope apparatus 101.

The internal insertion portion 1 is connected to the operation unit 3 disposed outside of the body or the guide portion 22 guiding the operation unit 3 through a number of ropes (elongated body) 51a, . . . which have flexibility and elasticity like rubber. This second embodiment describes an example in which there are four ropes 51.

One end of each of the four ropes 51a, . . . is connected to the base portion 9 of the internal insertion portion 1 which is disposed in the body cavity. The other ends of the two ropes 51a, 51b among the four ropes 51a, . . . extend from the internal insertion portion 1 in the lateral direction in the body cavity, and further extend to the outside of the body through the opening 2a made in the body wall 2. Then, the other ends of the two ropes 51a, 51b are turned back, and are connected to the operation unit 3 (See FIG. 4). The remaining other ends of the two ropes 51c, 51d among the four ropes 51a, . . . extend in the back and forth direction so as to become parallel to the rectilinear rails 23 in the body cavity, and further respectively extend to the outside of the body through the opening 2a made in the body wall 2. Then, the other ends of the two ropes 51c, 51d are turned back, and are connected to one end of the arched rail 24 via pulleys 52 disposed on the top of the arched rail 24.

As shown in FIG. 5 and FIG. 6, the operation unit 3 is provided with pulleys 53a, 53b for winding up or reeling out the ropes 51a, 51b, respectively. The other ends of the ropes 51a, 51b are fixed on the pulleys 53a, 53b respectively, and the other end of the rope 51a is wound around the pulleys 53a in the opposite direction to the other end of the rope 51b wound around the pulley 53b. When the operation unit 3 is positioned in the center of the arched rail 24, the lengths of the ropes 51a, 51b wound around the pulleys 53a, 53b are approximately half the length of the arched rail 24.

Also, as show in FIG. 6, the operation unit 3 is movable along an inner surface of the arched rail 24 by inserting upper end engaging parts 54a formed on both side portions 54 into arched grooves 24b formed on a side part of the arched rail 24. A shaft 55 is supported by side portions 54. A pinion 56 is supported by a shaft 55, and the pinion 56 is engaged on the teeth portion 24a formed on the inside of the arched rail 24. The pulleys 53a, 53b are attached to both ends of the pinion 56 so that the pulleys 53a, 53b rotate as a unit with the pinion 56. Thus, the pulleys 53a, 53b are rotated when the operation unit 3 is moved along the arched rail 24.

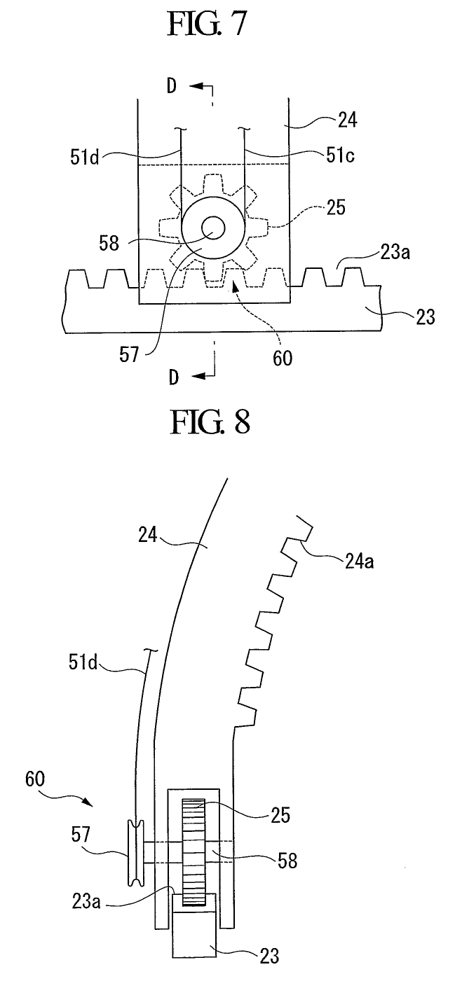

As shown in FIG. 7 and FIG. 8, a pulley 57 for winding up or reeling out the ropes 51a, 51b is disposed on an end of the arched rail 24. The other ends of the ropes 51c, 51d are fixed on the pulley 57, and the other end of the rope 51c is wound the pulley 57 in the opposite direction to the other end of the rope 51d wound around the pulley 57. When the operation unit 3 and the arched rail 24 are positioned in the center of the rectilinear rails 23, the length of the ropes 51c, 51d wound around the pulley 57 is approximately half the length of the rectilinear rails 23.

The pinion 25 which is engaged on the engage rack 23a of the rectilinear rail 23 is supported on a lower end of the arched rail 24 through a shaft 58, and the pulley 57 is attached to the shaft 58 so as to rotate as a unit with the pinion 25. Thus, the pulley 57 is rotated in a predetermined direction, when the arched rail 24 is moved along the rectilinear rails 23.

Note that, the pulley 57 is not restricted to just one, but two pulleys may be attached to the shaft 58 respectively corresponding to the ropes 51c, 51d. In this case, the pulleys are attached so as to rotate as a unit with the pinion 25.

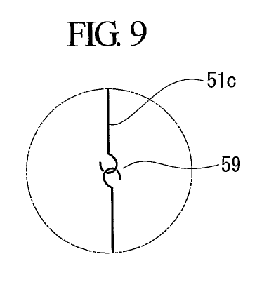

In addition, as shown in FIG. 9, the four ropes 51a, . . . each have hook-like connecting portions 59. The connecting portions 59 are detachable so as to be able to divide the rope at the mid-point.

In the second embodiment, the pulleys 23a, 23b, 57 and the pinions 25, 56 compose a length of rope adjusting portion 60 (length of elongated body adjusting portion) which adjusts the length of the ropes 51a, 51b, 51c, 51d. Further, the length of rope adjusting portion 60 and the ropes 51a, 51b, 51c, 51d compose the coupler 40 which moves the internal insertion portion 1 following the movement of the operation unit.

The operation of the endoscope apparatus 101 having the aforementioned configuration will be explained next.

The internal insertion portion 1 is inserted into the body cavity through one of the four openings 2a made in the body wall 2 while remaining the rope 51a outside the body. At this time, each of connecting portions 59 of the three ropes 51b, 51c, 51d which are inserted into the body cavity, is detached.

Next, ends of the three ropes 51b, 51c, 51d are removed from the body through the remaining three opening 2a, using rigid forceps or the like. The ends of the removed ropes 51b, 51c, 51d are attached to the other ends of the ropes 51b, 51c, 51d remaining in the operation unit 3 and the guide portion 22 through the connecting portions 59.

When the operation unit 3 is moved along the arched rail 24 manually, the pinion 56 which engages with the teeth portion 24a of the arched rail 24 is rotated, and the pulleys 53a, 53b are also rotated. According to this rotation, the rope 51a is wound up by the pulley 53a, and the rope 51b is reeled out from the pulley 53b. Therefore, the internal insertion portion 1 is pulled by the rope 51c wound up, and is moved in the body cavity coupling the operation unit 3.

Also, moving the operation unit 3 together with the arched rail 24 relative to the rectilinear rails 23 rotates the pinion 25 engaging with the rack 23a of the rectilinear rails 23, thereby rotating the pulley 57 accordingly. This rotation winds a rope 51c around the pulley 57 and feeds the another rope 51d therefrom. Therefore, the internal insertion portion 1 is pulled by the rope 51c wound up, and is moved in the body cavity coupling the operation unit 3.

When both movements are combined, since the ropes 51a, 51b, 51c, 51d, which have elasticity, are extended with a certain amount of the length, the length of the rope which is required is compensated.

Other operations are the same as those of the first embodiment.

According to the endoscope apparatus 101 as describe above, the internal insertion portion 1 and the operation unit 3 are coupled by the ropes 51a . . . so as to interpose the body wall 2 therebetween, and the operation unit 3 is moved along the body wall 2 outside the body, then the internal insertion portion 1 is moved following the operation unit 3. Therefore, it is possible to move the internal insertion portion 1 relative to the body wall 2 without restriction of the position of the opening 2a made in the body wall 2, and it is possible to observe the diseased part from any direction. Further, since the internal insertion portion 1 is coupled with the operation unit 3 so that the body wall 2 is interposed between the internal insertion portion 1 and the operation unit 3, it is easy to intuitively understand the position from which the image displayed on the monitor 8 is picked up, and it is easy to perform orientation. As a result, it is possible to treat the diseased part appropriately. Those advantages are the same as those of the first embodiment.

In the second embodiment, since the operation unit 3 is manually moved, and the motor is not used, the overall mechanism can be simplified, keeping costs low.

Note that, in the second embodiment, the operation unit 3 is manually moved. However, the operation unit 3 may be moved by using the driving force of the motor. Further, the endoscope apparatus may be provided with a zooming mechanism for enlarging the image of the imaging element 20 of the internal insertion portion 1, and a joint portion so as to perform angle adjustment of the observation portion main body relative to the base portion.

### Third Embodiment

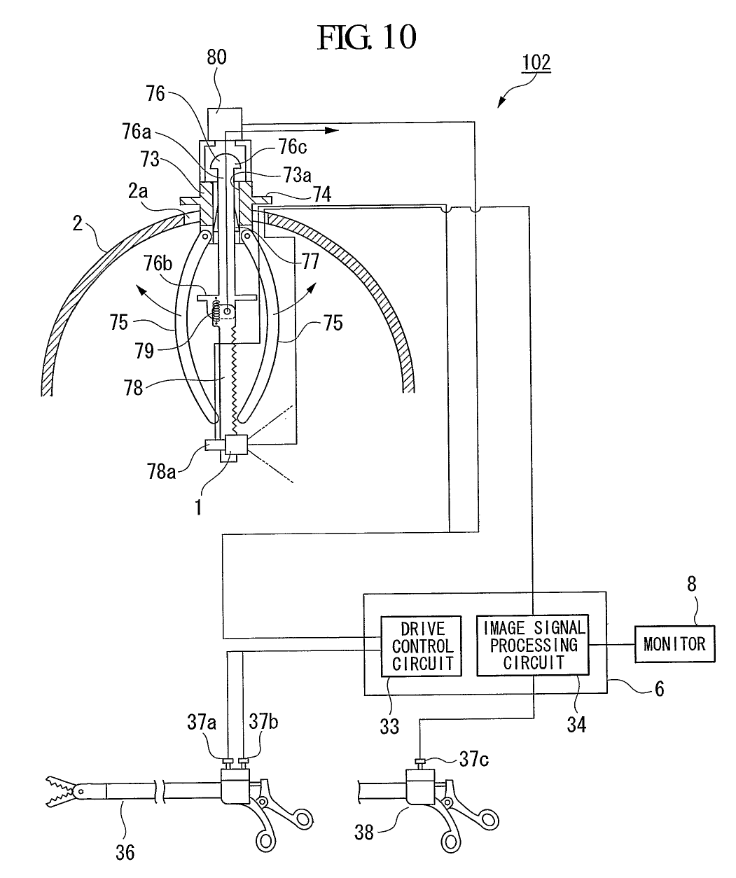

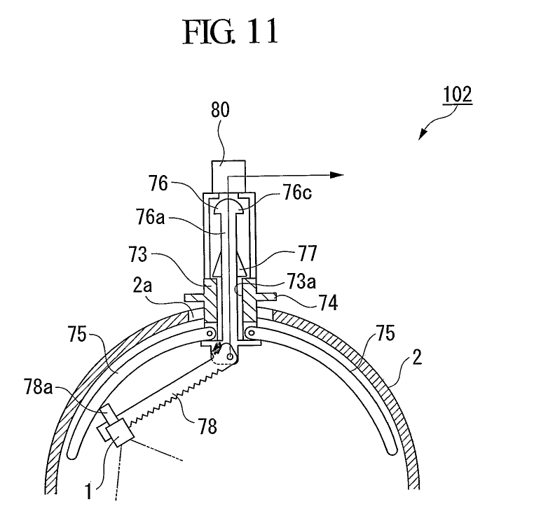

An explanation of the endoscope apparatus relating to the third embodiment of the present invention is described hereafter, with reference to FIG. 10 and FIG. 11. FIG. 10 is a view showing a state where a part of the endoscope apparatus is inserted into the body cavity, FIG. 11 is a view showing a state where the part of the endoscope apparatus has been inserted into the body cavity.

The third embodiment differs from the above-mentioned first embodiment as follows. That is, there is an internal rail 78, and a bone members 75 for moving the internal rail 78 smoothly in the third embodiment. As shown in those figures, there is the operation unit 3 outside the body in the third embodiment, as in the first and second embodiment. This point is omitted hereunder.

In the third embodiment, the numeral 73 indicates a cylindrical fixing member. A lower end thereof is inserted into the body cavity through the opening 2a made in the body wall 2 and is fixed. A flange portion 74 is formed on the operator's side of the fixing member 73. The diameter of the flange 74 is larger than that of the opening 2a made in the body wall 2. Thus, the insertion of the lower end of the fixing member 73 into the body cavity by more than a certain length is restricted.

A number of (for example, four or eight) the bone members 75 are attached to the lower end of the fixing member 73 so that those bone members 75 are arranged in the circumferential direction in a spaced manner, and are swingably arranged in a plane including a center axis of the fixing member 73. It is preferable that the bone member 75 is gently curved like the internal shape of the body wall 2.

A center rod 76 is inserted into a center hole 73a made in the fixing member 73. The center rod 76 is substantially rod-shaped except that both ends have a small diameter part 76a so as to be able to insert into the center hole 73a of the fixing member 73. Further, a large diameter part 76b is formed on the tip side (lower end side) of the center rod 76, and a handle 76c is formed on the operator's side (upper end side) of the center rod 76.

A shuttlecock portion 77 is disposed on the operator's side of the center rod 76. The shuttlecock portion 77 is spreadable by its own elasticity. When the shuttlecock portion 77 is closed, the center rod 76 can be put through the center hole 73a, when the shuttlecock portion 77 is spread, the center rod can not be put through the center hole 73a, because the shuttlecock portion 77 is spread larger than the diameter of the center hole 73a. Usually, the shuttlecock portion 77 is spread due to its own elasticity. The large diameter part 76b is larger than the diameter of the center hole 73a, and the diameter of the large diameter part 76b is substantially equal to that of a pin support portion of the fixing member 73, which swingably supports the bone members 75.

In addition, the length from the shuttlecock portion 77 to the large diameter part 76b is set to be approximately the same as the length of the center opening 73a of the fixing member 73.

An internal rail 78 is rotatably attached to a lower end of the center rod 76. The internal rail 78 is urged in a direction so that the internal rail 78 is bent relative to the center rod 78, by a spring 79 arranged between the internal rail 78 and the large diameter part 76b.

The internal insertion portion 1 is attached to the internal rail 78 so as to be able to move in a longitudinal direction of the internal rail 78. By a mechanism which is disposed between the internal rail 78 and the internal insertion portion 1 (for example, a mechanism formed by a rack and a pinion), the internal insertion portion 1 can be moved to an optional position, and can be fixed to the optional position. The internal insertion portion 1 is moved by, for example, a motor 78a which is built in the internal insertion portion 1. That is, the internal insertion portion 1 forms an internal guide portion which guides the internal insertion portion 1 in the body cavity. Further, a motor 80 which rotates the center rod 76 is disposed between the fixing member 73 and the center rod 76.

The operation of the endoscope apparatus 102 having the aforementioned configuration will be explained next.

The shuttlecock portion 77 is closed manually, and the center rod 76 is pushed into the center hole 73a of the fixing member 73. Further, while the bone members 75 are closed, the fixing member 73 is inserted into the body cavity through the opening 2a made in the body wall 2, until the flange portion 74 contacts the body wall 2.

Next, while the fixing member 73 is held manually, the center rod 76 is pulled up relative to the fixing member 73 until the shuttlecock portion 77 is exposed from the center hole 73a, and the large diameter part 76b contacts the fixing member 73. Accordingly, the fixing member 73 is interposed between the shuttlecock portion 77 and the large diameter part 76b by the shuttlecock portion 77 being spread by the elasticity thereof. As the result, the center rod 76 is fixed to the fixing member 73. Further, by the larger diameter part 76b being pulled up, a base portions of the bone members 75, then the bone members 75 are spread. Furthermore, the internal rail 78 is turned relative to the center rod 76 by a retracting force of the spring 79. In this state, it is possible to observe inside the body cavity.

At this time, the internal insertion portion 1 and operation unit (not shown) are positioned opposite each other so as to interpose the body wall 2 therewith.

While a picture image which is picked up by the imaging element of the internal insertion portion 1 is viewed using the monitor 8, the rigid forceps are inserted through the opening 2a which differs from the above-mentioned opening 2a, then the diseased part is treated. When it is required to observe the diseased part from a different angle, similar to the first embodiment, the operation unit (not shown) is moved by the switches 37a, 37b which are attached to the rigid forceps 36. The position of the operation unit is detected by a sensor (not shown), and a detection signal of the sensor is sent to the processor 6. A driving signal is sent from the drive control circuit 33 based on the positional signal of the operation unit. The motor 80 is driven by the driving signal, and rotates the center rod 76 until the internal insertion portion 1 corresponds to the operation unit. After that, the motor 78a is driven by the driving signal sent from the drive control circuit 33, thus the internal insertion portion 1 is moved relative to the internal rail 78. Therefore, it is possible to arrange the internal insertion portion 1 disposed inside of the body cavity on the position corresponding to the operation unit (not shown) so as to interpose a body wall 2 therewith.

Also after that, as the need arises, the zoom switch 37c disposed on the rigid forceps 38 is operated, and the diseased part is observed in an enlarged view.

When the treatment is finished, the motor 78a is driven by the driving signal from the drive control circuit 33, thus the internal insertion portion 1 is moved to the distal end of the internal rail 78. After that, the shuttlecock portion 77 of the center rod 76 is closed manually, and the center rod 76 is pushed into the fixing member 73. The bone members 75 lose support from the large diameter part 76b of the fixing member 73, and change from the spread state to the closed state. At the same time, the internal rail 78 is also pushed by the bone members 75, and an axis of the internal rail 78 matches that of the center rod 76. In this state, the center rod 76 is pulled out from the opening 2a of the body wall 2, with the fixing member 73.

According to the endoscope apparatus 102 as described above, the internal insertion portion 1 can be moved along the internal rail 78, which extends in any directions, and can be turned. Therefore, it is possible to move the internal insertion portion 1 relative to the body wall 2 without restricting the position of the opening 2a made in the body wall 2, and it is possible to observe the diseased part from any direction. Further, since the internal insertion portion 1 is coupled with the operation unit 3 so that the body wall 2 is interposed between the internal insertion portion 1 and the operation unit 3, it is easy to intuitively understand the position from which the image displayed on the monitor 8 is picked up, and it is easy to perform orientation. As a result, it is possible to treat the diseased part appropriately. Those advantages are the same as those of the first embodiment.

Additionally, in the third embodiment, the internal insertion portion 1 is moved inside of the body cavity by the internal rail 78 and the center rod 76, which can be turned, in other words, the internal insertion portion 1 is moved in the vicinity of the polar coordinate. Therefore, it is possible to move the internal insertion portion 1 smoothly. Further, since the bone members 75 are spread inside of the body cavity, it is unnecessary to perform aeroperitoneum by generally using CO₂ gas. As a result, it is possible to provide the operation space inside of the body cavity.

### Fourth Embodiment

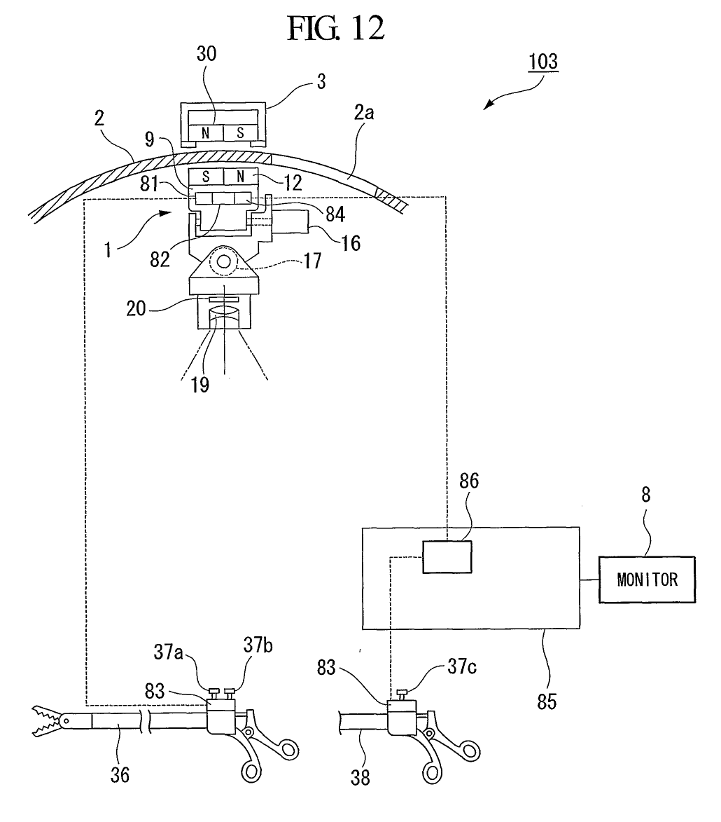

An explanation of an endoscope apparatus 103 relating to the fourth embodiment of the present invention is described hereafter, with reference to FIG. 12. FIG. 12 is a view showing a state where the part of the endoscope apparatus has been inserted into the body cavity.

The fourth embodiment differs from the above-mentioned first embodiment as follows. That is, the endoscope apparatus 103 does not include the guide portion 22, and the operation unit 3 is directly moved on the body wall 2. Further, the internal insertion portion 1 is provided with a receiving portion 81 and a driving processor 82, and the rigid forceps 36, 38 are provided with a transmitting portion 83. Furthermore, the internal insertion portion 1 is provided with a transmitting portion 84, and an image processor 85 which is disposed outside of the body is provided with a receiving portion 86.

That is, for example, the receiving portion 81, the driving processor 82 and receiving portion 84 are built in the base portion 9 of the internal insertion portion 1. The receiving portion 81 receives a signal sent from the transmitting portion 83 of the rigid forceps 36. Further, an image signal obtained by the imaging element 20 is subjected to a predetermined process, and is sent to the receiving portion 86 of the image processor 85. The receiving portion 86 of the image processor 85 also receives a signal which is sent from the transmitting portion 83 connected to the zoom switch 37c of the rigid forceps 38.

Next, an explanation of the operation of the above composed endoscope apparatus 103 according to the fourth embodiment is provided.

The internal insertion portion 1 is inserted inside of the body cavity through the opening 2a made in the body wall 2, and the operation unit 3 is arranged outside of the body wall 2. Thus, the internal insertion portion 1 is attached to the inner surface of the body wall 2 corresponding to the operation unit 3 by the magnetic force 12, 30. Then, the operation unit 3 is held manually, and the operation unit 3 is moved along the body wall 2 in the back and forth directions, and in the right and left directions. Thus, it is possible to synchronously move the internal insertion portion 1 to an optional position inside of the body cavity, by the magnetic force 12, 30.

When the movement is finished, the operation unit 3 is released. However, the internal insertion portion 1 is fixed on the position by an interposing force which acts between the internal insertion portion 1 and the operation unit 3 by the magnetic force 12, 30.

Image zooming and angle changing are performed as follows. That is, by the switches 37a, 37b which are attached to the rigid forceps 36 being operated, a signal is sent to the receiving portion 81 disposed inside of the internal insertion portion 1 through the transmitting portion 83 by wireless, thus the motors 16, 17 are controlled through the driving processor 82.

Similarly, an image inside of the body cavity which is obtained by the internal insertion portion 1 is sent to the receiving portion 86 of the image processor 85 through the transmitting portion 84 disposed inside of the internal insertion portion 1, is then displayed by the monitor 8. When it is required to enlarge the image, by the switch 37c which is attached to the rigid forceps 38 being operated, a signal is sent to the receiving portion 86 of the image processor 85 through the transmitting portion 83 by wireless. An image signal is subjected to a predetermined process, and an enlarged image is displayed by the monitor 8.

According to the endoscope apparatus 103 as described above, the internal insertion portion 1 can be supported by the operation unit 3 using an attaching force of the magnets 12, 30 so as to interpose the body wall 2 therewith. Therefore, it is possible to move the internal insertion portion 1 relative to the body wall 2 without restriction of the position of the opening 2a made in the body wall 2, and it is possible to observe the diseased part from any direction. Furthermore, since the internal insertion portion 1 is coupled with the operation unit 3 so that the body wall 2 is interposed between the internal insertion portion 1 and the operation unit 3, it is easy to intuitively understand the position from which the image displayed on the monitor 8 is picked up, and it is easy to perform orientation. As a result, it is possible to treat the diseased part appropriately. Those advantages are the same as those of the first embodiment.

Additionally, in the fourth embodiment, there is no guide portion 22 which guides the operation unit 3 such as the rectilinear rail and the arched rail. Accordingly it is possible to downsize the device, and to reduce costs. In addition, it is possible to easily prepare setting the device.

### Fifth Embodiment

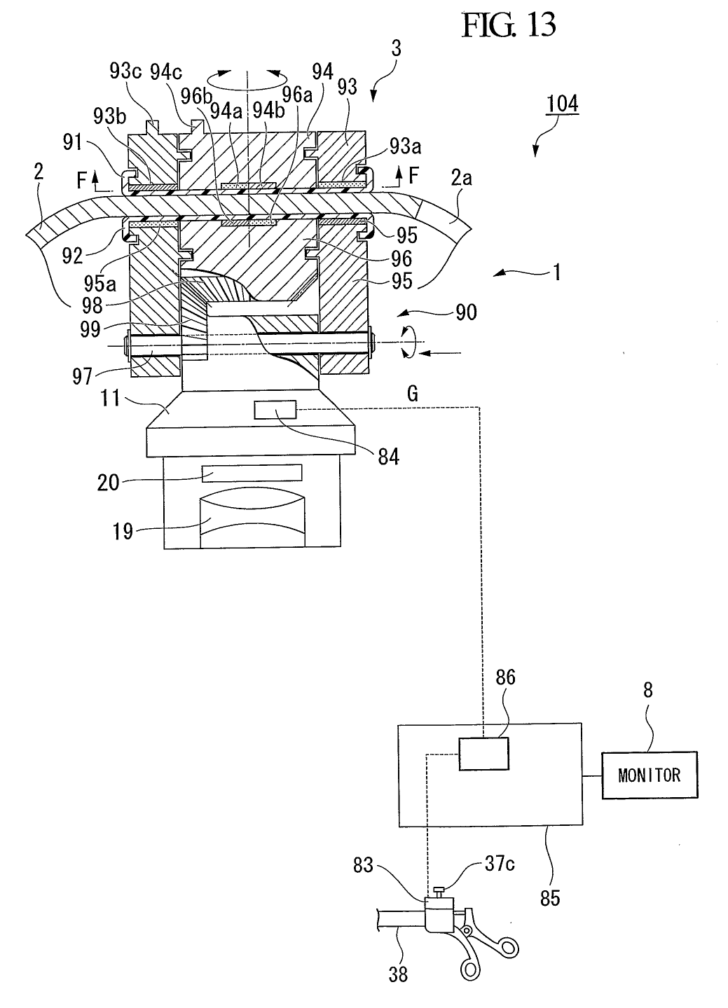

Fifth embodiment of the present invention will be explained with reference to FIGS. 13 to 15. FIG. 13 is a view showing a state where a part of the endoscope apparatus has been inserted into the body cavity.

The fifth embodiment is different from the fourth embodiment in that rotating force of a inward-rotating plate 94 disposed to the operation unit 3 is transmitted to the observation unit main body 11 through a driving-force-converting portion 90 in the internal insertion portion 1, thereby changing the inclination angle of the observation unit main body 11. Also, slide plates 91 and 92 are placed among the operation unit 3, the body wall 2, and the internal insertion portion 1.

That is, the operation unit 3 includes the slide plate 91, a ring-shaped outward-rotating plate 93 disposed on upper portion of the slide plate 91, and an inward-rotating plate 94 attached inside of the outward-rotating plate 93. The slide plate 91, the outward-rotating plate 93, and the inward-rotating plate 94 are coaxially rotative relative to each other. The rotational axes lines of the outward-rotating plate 93 and the inward-rotating plate 94 coincide with an axial line connecting the operation unit 3 and the internal insertion portion 1.

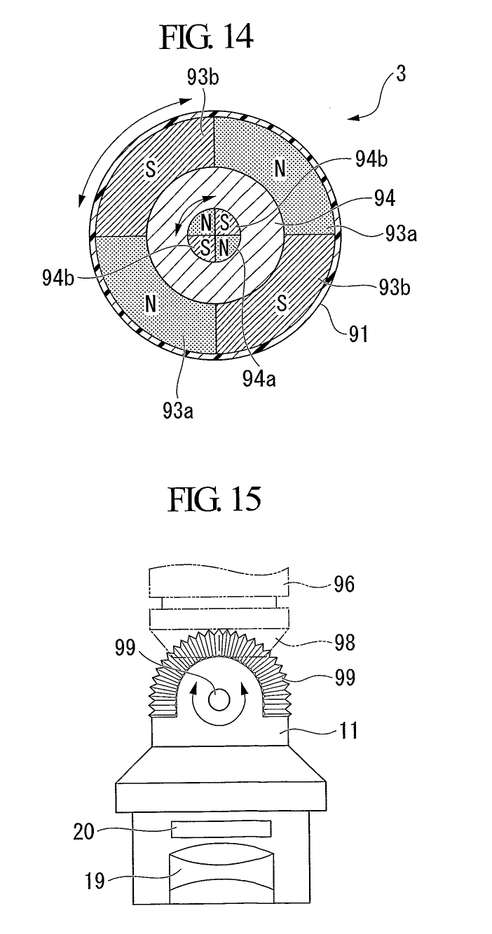

As shown in FIG. 14, provided on a bottom surface of the outward-rotating plate 93 are equiangular magnets 93a having north poles and equiangular magnets 93b having south poles that are alternately disposed in the circumference direction. Provided also on a bottom surface of the inward-rotating plate 94 are equiangular magnets 94a having north poles and equiangular magnets 94b having south poles that are alternately disposed in the circumference direction. Incidentally, a silicon resin or a fluorine resin may be a non-magnetic slide plate 91 having insignificant coefficient of friction is free of detrimental effect on human bodies. Also, the outward-rotating plate 93 and the inward-rotating plate 94 may be made from a non-metallic material that is free of detrimental effect on human bodies. Reference numeral 93c indicates a handle integrated with the outward-rotating plate 93. Reference numeral 94c indicates a handle integrated with the inward-rotating plate 94.

On the other hand, the internal insertion portion 1 includes: the slide plate 92; a cylindrical casing 95 disposed beneath the slide plate 92; a rotating member 96 disposed inside the casing 95; and the observation unit main body 11 rotatively supported beneath the rotating member 96 by means of a shaft 97 attached to the casing 95 in a direction orthogonal to the slide plate 92. The slide plate 92, the casing 95, and the rotating member 96 are coaxially rotative relative to each other.

Provided on an upper surface of the casing 95 are equiangular magnets 95b having north poles on their upper surfaces and equiangular magnets 95a having south poles on their upper surfaces that are alternately disposed in the circumference direction. The magnets 95b and 95a correspond in number and size to the magnets 93a and 93b attached on the bottom surface of the outward-rotating plate 93. That is, the number is the same and the size is set to be approximately the same. Provided also on an upper surface of the rotating member 96 are equiangular magnets 96b having north poles on their upper surfaces and equiangular magnets 96a having south poles that are alternately disposed in the circumference direction. The magnets 96b and 96a correspond in number and size to the magnets 94a and 94b attached on the bottom surface of the inward-rotating plate 94.

The surface having the outward-rotating plate 93 and the inward-rotating plate 94 of the operation unit 3 faces to the surface having the casing 95 and the rotating member 96 of the internal insertion portion 1, and the magnets disposed on the surfaces constitute a rotational force transmission section that contactlessly transmits the rotational force of the operation unit 3 to the internal insertion portion 1.

The example shown in the drawing is not limited to two magnets facing two magnets on the facing surfaces, that is, one magnet may face to one magnet, or three or more magnets may face to the corresponding number of magnets. Although the drawing shows the example in which the magnets disposed on the outward-rotating plate 93 (of the casing 95) are the same as the magnets disposed on the inward-rotating plate 94 (of the rotating member 96) in number, i.e., two pieces on the north pole and two pieces on the south pole, four pieces in total, the number may not necessarily be the same. That is the number of magnets may be variable. Note that the magnets may be the same in number, and alternatively different in number.

Formed beneath the rotating member 96 is a bevel gear 98. The bevel gear 98 engages with a bevel gear 99 disposed to the observation unit main body 11. That is, the bevel gears 98 and 99 constitute the driving-force-converting portion 90 for transmitting the rotation of the rotating member 96 linking to the rotation of the inward-rotating plate 94 of the operation unit 3 to the observation unit main body 11 so as to rotate the observation unit main body 11 around an axis 97. Provided further to the observation unit main body 11 is a transmitting portion 84 for carrying out a predetermined process on an image data obtained by an imaging element 20 and sending it to a receiving portion 86 of an image processor 85.

An operation of an endoscope apparatus 104 according to fifth embodiment will be explained.

Absorbing forces produced by the magnets 93a, 95b, 93b, and 95a of the outward-rotating plate 93 and the casing 95 facing to each other and absorbing forces produced by the magnets 94a, 94b, 96b, and 96a of the inward-rotating plate 94 and the rotating member 96 cause the internal insertion portion 1 to stick to the inner surface of the body wall corresponding to the operation unit 3 by inserting the internal insertion portion 1 through the opening 2a formed in the body wall 2 and disposing the operation unit 3 outside of the body wall 2.

The internal insertion portion 1 may be inserted through a natural orifice of a human body and through an opening formed in the wall of a tissue to be disposed at a desired position without forming the opening 2a in the body wall 2. For example, it may be inserted through an opening formed in a gastric wall and disposed at a desirable location.

Then, the operation unit 3 is held manually, and the operation unit 3 is moved along the body wall 2 in the back and forth and around. Thus, it is possible to synchronously move the internal insertion portion 1 to an optional position inside of the body cavity in connection with the absorbing force produced by the magnets. The slide plates 91 and 92 having insignificant coefficient of friction placed between the magnets and the body wall 2 provide smooth movements to the operation unit 3 and the internal insertion portion 1 compared with a case in which the inward-rotating plate 94 and the casing 95 move while making direct contact to the body wall 2.

Although the hand appended to the operation unit 3 is detached upon finishing the movement to the predetermined position, the internal insertion portion 1 can be held at its current position by the absorbing force of the magnets disposed on the surfaces facing between the internal insertion portion 1 and the operation unit 3.

Adjusting the inclination angle of the observation unit main body 11 of the internal insertion portion 1 requires first rotating the outward-rotating plate 93 together with the inward-rotating plate 94 of the operation unit 3. This accordingly rotates the casing 95 absorbed to the outward-rotating plate 93 by magnets and the rotating member 96 absorbed to the inward-rotating plate 94 by magnets according to the outward-rotating plate 93, etc. That is, the internal insertion portion 1 also rotates in accordance with the rotation of the operation unit 3. The outward-rotating plate 93 and the casing 95 thus realizes smooth rotation because the slide plates 91 and 92 making direct contact with the body wall 2 are fixed, i.e., makes no rotation.

After determining the direction of the observation unit main body 11 of the internal insertion portion 1, only the inward-rotating plate 94 is rotated while manually holding the outward-rotating plate 93. The casing 95 absorbed by the outward-rotating plate 93 by the magnets 93a, 93b, 95b, and 95a facing to each other does not rotate. On the other hand, the rotating member 96 absorbed by the inward-rotating plate 94 by the magnets 94a, 94b, 96b, and 96a facing to each other rotates with the inward-rotating plate. This rotates the rotating member 96 relative to the casing 95, and its rotational force transmitted through the bevel gears 98 and 99 further rotates the observation unit main body 11 around the axis 97 by a predetermined angle. Eventually, the observation unit main body 11 can be rotated by a predetermined angle around the axis 97 in accordance with the revolution of the inward-rotating plate 94, and accordingly the inclination angle of the observation unit main body 11 can be adjusted.

The image inside of the body cavity which is captured by the internal insertion portion 1 is sent to the receiving portion 86 of the image processor 85 through the transmitting portion 84 disposed inside of the internal insertion portion 1, is then displayed by the monitor 8. When it is required to enlarge the image, by the switch 37c which is attached to the rigid forceps 38 being operated, a signal is sent to the receiving portion 86 of the image processor 85 through the transmitting portion 83 by wireless. An image signal is subjected to a predetermined process, and an enlarged image is displayed by the monitor 8.

According to the endoscope apparatus 104 as described above, the internal insertion portion 1 can be supported by the operation unit 3 using an attaching force of the magnets so as to interpose the body wall 2 therewith. Therefore, it is possible to move the internal insertion portion 1 relative to the body wall 2 without restriction of the position of the opening 2a made in the body wall 2, and it is possible to observe the diseased part from any direction. Furthermore, since the internal insertion portion 1 is coupled with the operation unit 3 so that the body wall 2 is interposed between the internal insertion portion 1 and the operation unit 3, it is easy to intuitively understand the position from which the image displayed on the monitor 8 is captured, and it is easy to perform orientation. As a result, it is possible to treat the diseased part appropriately. Those advantages are the same as those of the first and fourth embodiments.

In addition, manipulating the operation unit 3 enables the direction and the inclination angle of the observation unit main body 11 to be adjusted in the fifth embodiment. Since the guide portion 22 like rectilinear rails and arched rails are not necessary, and in addition, driving units like motor are not necessary in the internal insertion portion 1, the internal insertion portion 1 may be smaller with reduced costs.

While preferred embodiments of the invention have been described and illustrated above, it should be understood that these are exemplary of the invention and are not to be considered as limiting. Modifications can be made without departing from the spirit or scope of the present invention.

For example, in the first and fourth embodiments, for attaching the operation unit 3 and the internal insertion portion to each other, the magnets 12, 30 are respectively disposed on the operation unit 3 and the internal insertion portion so as to face each other. However, one of the magnet 12 or the magnet 30 may be replaced with a magnetic body.

Instead of the above embodiments in which the imaging element 20 is built in the internal insertion portion 1, a treating section for carrying out a predetermined treatment to a diseased part may be provided. The imaging element 20 may also be provided in this alternative case.

For example, in the first and fourth embodiments, for attaching the operation unit 3 and the internal insertion portion to each other, the magnets 12, 30 are respectively disposed on the operation unit 3 and the internal insertion portion so as to face each other. However, one of the magnet 12 or the magnet 30 may be replaced with a magnetic body.

The internal insertion portion 1 according to the above fifth embodiment is not limited to the configuration in which the outward-rotating plate 93 of the operation unit 3 is used for fixing the casing 95, and the inward-rotating plate 94 is used for rotating the rotating member 96. To the contrary, the casing 95 and the rotating member 96 may be reversed in position, i.e., the outward-rotating plate 93 of the operation unit 3 may be used for rotating the rotating member 96, and the inward-rotating plate 94 may be used for fixing the casing 95.

Also, the fifth embodiment may have a configuration in which an detachable engaging section may be disposed between the outward-rotating plate 93 of the operation unit 3 and the inward-rotating plate 94 so that the engaged state of the engaging section rotates the rotating plate 93 together with the rotating plate 94; and released state of the engaging section rotates one of the plates relative to the other one.

In the present embodiment, it is possible to synchronously move the operation unit 3 and the internal insertion portion 1 by the magnetic force. However, the magnetic force may be used only for fixing the internal insertion portion 1 to an inner surface of the body wall, and the internal insertion portion 1 may be immovably fixed inside of the body cavity.

In this case, the object optical system 19 of the internal insertion portion 1 is provided with a wide-angle optical system which is able to capture the entire area of the body cavity in sight, and the monitor 8 displays only one part of an image which is captured. Also, an area which is desired to be displayed by the monitor 8 can be moved in the back and forth directions, and in the right and left directions using an inputting device secured to the operation unit 3, and the area can be specified. Therefore, any desirable area can be displayed by the monitor 8 among the entire image area of the object optical system 19. By doing this, it is possible to change the view.

In this case, the internal insertion portion 1 is not only fixed to the inner surface of the body wall by the magnetic force, but the internal insertion portion 1 is also fixed to the inner surface of the body wall by a different mechanical device.

Further, it is conceivable to secure the view by the internal insertion portion 1 being moved freely, without fixing the internal insertion portion 1 to the inner surface of the body wall.

For example, liquid such as water or saline is filled in the body cavity, and the internal insertion portion 1 is arranged in the liquid. Then, the internal insertion portion 1 can be moved in the liquid by a signal which is sent from outside of the body. Note that, a buoyant force acts on the internal insertion portion 1 arranged in the liquid. When the liquid is moved, a reaction force is caused, and the internal insertion portion 1 can be moved by the reaction force. Therefore, it is advantageous that the internal insertion portion 1 is arranged in the liquid.

In detail, the internal insertion portion 1 is provided with a device for obtaining thrust such as a propeller, and the device may be controlled by wireless outside of the body. Further, by balancing the magnetic force acting from outside of the body cavity with the weight of the internal insertion portion 1, the distance between the internal insertion portion 1 and the diseased part can be adjusted. Furthermore, by moving the magnetic force generator, a magnetic field outside of the body cavity changes. Therefore, it is possible to move the internal insertion portion 1.

1.-11. (canceled)

12. An endoscope apparatus comprising:
an endoscope which has an imaging element and
a monitor which displays an image imaged by the imaging element, wherein
the endoscope is provided with an internal insertion portion which is movably arrangeable inside a body cavity, and an operation unit for changing the position of the internal insertion portion, which is arrangeable outside the body, and
the internal insertion portion and the operation unit are arranged on a common axis so as to interpose a body wall therebetween, wherein
the endoscope apparatus further comprises
a rotational force transmission section for contactlessly transmitting the rotational force of the operation unit produced around an axial line between the internal insertion portion and the operation unit to the internal insertion portion.

13. The endoscope apparatus according to claim 12, wherein the rotational force transmission section comprises magnets correspondingly disposed on the surface having the operation unit and on the surface having the internal insertion portion, and
a non-magnetic slide plate is disposed in the space between the magnets and the body wall.

14. The endoscope apparatus according to claim 12, wherein an optical axis of an observational optical system accompanying the imaging element is disposed diagonally with respect to the axial line between the internal insertion portion and the operation unit.

15. An endoscope apparatus comprising:
an endoscope which has an imaging element and
a monitor which displays an image imaged by the imaging element, wherein
the endoscope is provided with an internal insertion portion which is movably arrangeable inside a body cavity, and an operation unit for changing the position of the internal insertion portion, which is arrangeable outside the body, and
the internal insertion portion and the operation unit are arranged on a common axis so as to interpose a body wall therebetween, wherein
the operation unit has a rotating section for rotating around an axial line between the operation unit and the internal insertion portion, and
the internal insertion portion has a rotative member for contactlessly receiving the rotational force of the rotating section of the operation unit; and
a driving-force-converting portion for converting the revolution of the rotating section into the rotation around an axis orthogonal to the axial line and transmitting the converted rotation to a portion including the imaging element.

16. The endoscope apparatus according to claim 12, wherein
the operation unit has a rotating section for rotating around an axial line between the operation unit and the internal insertion portion, and
the internal insertion portion has a rotative member for contactlessly receiving the rotational force of the rotating section of the operation unit; and a driving-force-converting portion for converting the revolution of the rotating section into the rotation around an axis orthogonal to the axial line and transmitting the converted rotation to a portion including the imaging element.

17. An endoscope apparatus comprising:
an endoscope which has an imaging element and
a monitor which displays an image imaged by the imaging element, wherein
the endoscope is provided with an internal insertion portion which is movably arrangeable inside a body cavity and an operation unit for changing the position of the internal insertion portion, which is arrangeable outside the body, and
the internal insertion portion and the operation unit are arranged on a common axis so as to interpose a body wall therebetween, wherein
the endoscope apparatus further comprises slide plates disposed among the rotating section of the operation unit, the rotative member of the internal insertion portion, and the body wall.

18.-19. (canceled)
